(19) United States
(12) Patent Application Publication
(10) Pub. No.: US 2010/0323434 A1
(43) Pub. Date: Dec. 23, 2010

(54) Compartmentalized Nerve Culture System

(76) Inventor: In Hong Yang, Baltimore, MD (US)

Correspondence Address: STEIN MCEWEN, LLP, 1400 EYE STREET, NW, SUITE 300, WASHINGTON, DC, 20005, US

(21) Appl. No.: 12/817,192

(22) Filed: Jun. 17, 2010

Related U.S. Application Data
(60) Provisional application No. 61/218,444, filed on Jun. 19, 2009.

(51) Int. Cl. C12M 3/00

(52) U.S. Cl. 435/289.1

(57) ABSTRACT

The present invention relates generally to a compartmentalized nerve culture system. The compartmentalized nerve culture system has numerous applications including isolation of axons and cell bodies. The present invention has broad application for basic and pre-clinical research including, but not limited to, use in neuroscience, neuronal culture systems, co-culture systems, drug screening, morphological studies, and toxicology studies.

### CROSS REFERENCE TO RELATED APPLICATIONS

This application claims priority to U.S. Provisional Patent Application Ser. No. 61/218,444, entitled “Compartmentalized Nerve Culture System” which was filed Jun. 19, 2009, the entire contents of which are incorporated herein by reference.

### FIELD OF THE INVENTION

The present invention relates generally to a compartmentalized nerve culture system. The compartmentalized nerve culture system has numerous applications including isolation of axons and cell bodies. The present invention has broad application for basic and pre-clinical research including, but not limited to, use in neuroscience, neuronal culture systems, co-culture systems, drug screening, morphological studies, and toxicology studies.

### BACKGROUND OF THE INVENTION

The mammalian central nervous system (CNS) is composed of several highly specialized cell types, including neurons and glial cells. Neurons communicate with each other via dendrites and axons, which respectively are responsible for integrating synaptic input and providing output. CNS axons, and to a lesser extent dendrites, often span substantial distances and are situated in compositionally distinct microenvironments as compared to their cell bodies. Axonal degeneration and axon-glia interactions have been strongly implicated in the pathogenesis of a growing number of neuroinflammatory and neurodegenerative diseases, but the underlying cellular and molecular mechanisms have yet to be identified.

Elucidation of mechanisms involved in axonal injury and axon-glia interactions can be difficult in the complex in vivo setting, and thus many investigators have turned to in vitro approaches. However, standard cell culture approaches do not compartmentalize axons from neuronal cell bodies, thus making it difficult to delineate axon-specific mechanisms. To address this problem, the Campenot chamber, which enables manipulation of axons independently from neuronal cell bodies, was developed. Campenot chambers consist of a Teflon divider attached to collagen-coated Petri dish with silicone grease. Campenot chambers require great skill, as leakage between chambers is a common problem, limiting efficiency and reproducibility. Studies using the Campenot chamber have led to discoveries in peripheral nervous system (PNS) axonal development, degeneration, and regeneration. However, the far smaller sizes of CNS neurons and axons have precluded the reproducible study of compartmentalized CNS neuronal cultures within Campenot chambers.

Recently, it has been recognized that lab-on-a-chip devices may provide unique solutions to study cellular and molecular aspects of neuronal and axonal function, with precise control over the cellular microenvironment. In particular, microchannels have been shown to be powerful micro-features capable of passively guiding axons of both CNS and PNS neurons. Several novel microfluidic devices have utilized these features to fluidically isolate axons from their respective cell bodies. In these devices, cells are introduced by way of a loading inlet and the cells are randomly dispersed throughout a cell reservoir. Neuronal cells that are in close proximity to the microchannels extend axons through the channels and into adjacent compartments. Application of hydrostatic pressures between microchannel-connected compartments can induce fluidic isolation between axon and cell body.

Conventional microfluidic approaches are, however, limited by the inability to precisely place cells within microscopic areas of interest and to control neuronal and axonal microenvironments. There has thus been a long-felt need for improved microfluidic approaches.

### SUMMARY OF THE INVENTION

The present invention relates generally to a compartmentalized nerve culture system. One object of the invention is to provide a compartmentalized microfluidic system, comprising a base substrate; a plurality of microchannels; a plurality of somal compartments; and a plurality of axonal compartments, wherein the microchannels are formed within the base substrate, and further wherein the microchannels are connected with the somal compartments and the axonal compartments.

Further objects and advantages of the invention will become apparent from a consideration of the drawings and ensuing description.

### BRIEF DESCRIPTION OF THE FIGURES

FIG. 1 depicts fabrication of a co-culture platform according to one embodiment of the present invention.

FIG. 2A depicts a schematic diagram of a PDMS device according to one embodiment of the present invention, showing the microchannels and somal and axonal compartments.

FIG. 2B depicts large compartments that enable pipetting close to the microchannels.

FIG. 3 A depicts a device configured so that it consists of multiple somal compartments connected to a unified axonal compartment.

FIG. 3 B depicts primary microglial cells selectively placed between bundles of axon outgrowth in the unified axonal compartment through the use of PDMS microstencils.

### DETAILED DESCRIPTION OF THE PREFERRED EMBODIMENTS

It is to be understood that all references cited herein are incorporated by reference in their entirety.

The invention relates generally to a compartmentalized nerve culture system (hereinafter, “nerve culture system”).

According to a preferred embodiment of the invention, the compartmentalized nerve culture system is designed as a compartmentalized microfluidic system for the study of axon-glia interactions. In another preferred embodiment of the present invention, a poly(dimethylsiloxane) (PDMS)-based microfluidic system is capable of isolating large axonal populations within a small fluidic volume. The isolation is achieved by the compartmentalized microfluidic chamber which includes arrays of microchannels that topographically guide growing axons near grooved interfaces in the chamber.

The novel microfluidic system described herein advances existing device technologies to enable directed placement of neurons and glial cells within defined microenvironments in a compartmentalized microfluidic platform. In a preferred embodiment, the circular microfluidic device consists of multiple independent units arranged in a closed circular pattern, with each unit consisting of two compartments (somal and axonal) connected by an array of microchannels. Fluidic access ports are punched on both somal and axonal sides near the microchannels, allowing for direct pipetting of cells close to the microchannel interface. During the creation of fluidic access ports, adjacent somal or axonal compartments can be readily merged so that independent groups of neurons or axons can be maintained in either distinct or uniform microenvironments.

The present invention advantageously provides different modes of directed cell placement in the device, to suit varying experimental needs for the study of axon-glia interactions. First, the circular geometry of the platform lends itself to centrifugation, or spinning, to optimize the positioning of both neurons and glial cells, enhancing axonal throughput in microchannels and providing a new technique to establish axon-glia interactions. Second, patterned microstenciling can be utilized to directly place glial cells within areas of interest in the axonal compartment. Third, intimate axon-glia co-culture can be attained via standard pipetting techniques as a result of the close proximity of large access ports to the microchannel interface.

To demonstrate the diverse capabilities of the novel microfluidic platform, we investigated whether microglia specifically respond to injured axons in the setting of CNS inflammatory disease. Microglial accumulation to sites of neurodegeneration plays a major role in maintenance and progression of a number of chronic neuroinflammatory and neurodegenerative diseases, but it is not known whether microglia are specifically recruited toward degenerating CNS axons in the absence of signals from other neural cells. To address this question, the novel device described herein was configured to have multiple somal compartments whose microchannels connect to a unified axonal compartment, thereby creating independent populations of neurons whose axons reside in a common microenvironment. By placing microglia in spatially defined areas between groups of axons, we were able to monitor microglial responses to populations of axons that were either healthy or degenerating. To our knowledge, this is the first demonstration of a differential microglial response to healthy versus injured CNS axons in a microfluidic platform.

The compartmentalized microfluidic platform according to the present invention can be designed and fabricated in accordance with any desired size, shape and dimensions. In a preferred embodiment, the microfluidic platform is generally circular in shape.

The compartmentalized circular microfluidic platform advantageously enables directed cell placement within defined microenvironments for the study of axon-glia interactions. Microfabrication technology, utilizing photolithography, micro-contact printing, and microfluidics, is preferably used to construct the chambers according to the present invention, in which precise in vitro cellular patterning is achieved.

The compartmentalized microfluidic system of the present invention provides several additional surprising and unexpected advantages. Surprisingly, the compartmentalized system, in which all compartments are open to air, makes it easy to load cells into the system. There is also unexpectedly good gas exchange; and the compartmentalized system also makes it significantly easier for immunostaining experiments. There is also surprisingly better media buffering for cell culture because all the compartments of the nerve culture system of the present invention are open to air. Also, additional advantages include a lack of buffering problems, since each compartment can hold a substantial amount of media, e.g., preferably more than about 0.5 ml of media. Also, the nerve culture system of the present invention can be easily designed for high-throughput applications. For instance, the system can easily be designed with more than 500 microchannels per platform, and even 2500 or more microchannels per platform can easily be obtained with the present inventkion. The nerve culture system of the present invention also enables higher occupation of microchannels with axons which offers several unexpected advantages, including benefits in myelination experiments, since a higher density of axons is important in myelination experiments. Moreover, the nerve culture system of the present invention also makes it surprisingly easy to align cells close to the microchannels; for instance, cells can be placed into microchannels by pipette or micro-pipette techniques. The present invention offers additional numerous advantages for analyzing cell-cell interactions, including the formation of cellular junctions, since different types of cells can be placed in the open-air compartments of the compartmentalized microfluidic system.

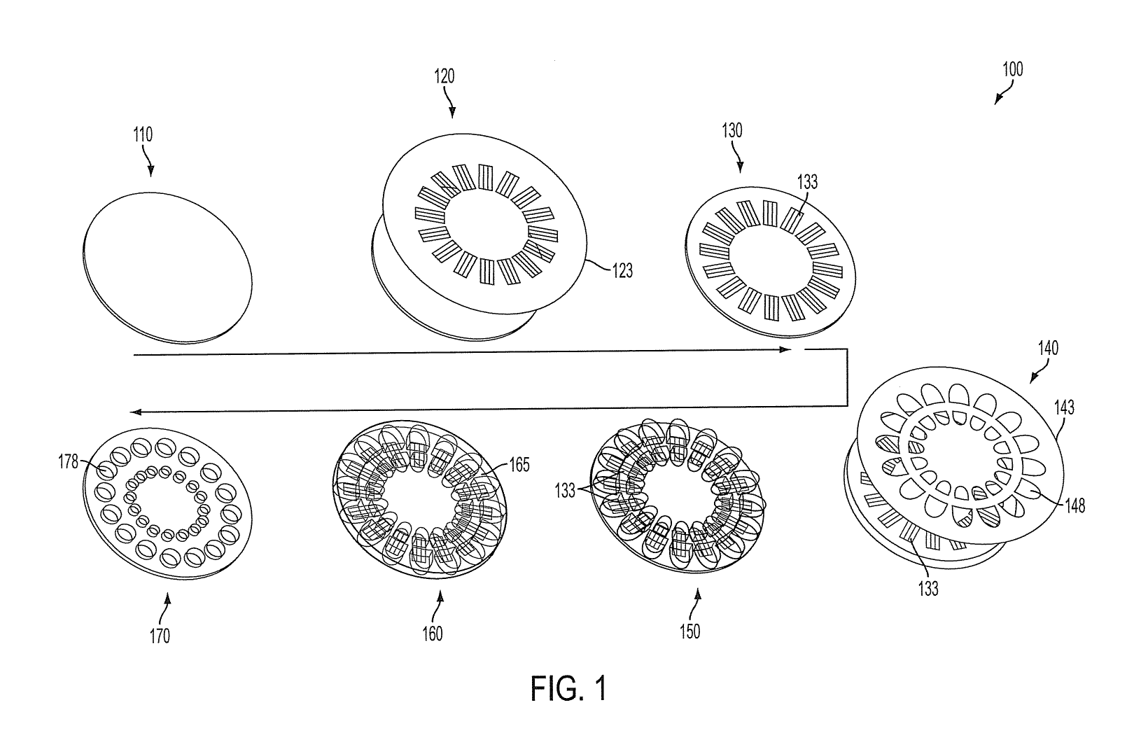

With reference to FIGS. 1, 2 and 3, as described herein, exemplary embodiments of the compartmentalized microfluidic system according to the present invention are shown. The compartmentalized microfluidic system can be easily fabricated and used to separate the sub-cellular parts of the neuron, and it can be built preferably with sub-micron resolution using soft lithography and glass etching techniques; and can be designed in any shape and size as needed or desired, including for example circular and linear shaped systems.

With the present invention, there are provided numerous distinct possible modes of directed cell placement for placing cells in the compartmentalized system, to suit varying experimental needs for the study of axon-glia interactions. In one embodiment, centrifugation of the circular platform can result in a two-fold increase in axonal throughput in microchannels and provides a new technique to establish axon-glia interactions. According to another embodiment, microstencils can be utilized to directly place glial cells within areas of interest. According to yet another embodiment, intimate axon-glia co-culture can be attained via pipetting techniques. One can take advantage of the microfluidic platform of the present invention to study neuronal cells in culture. For instance, the present invention has been used to demonstrate a two-fold preferential accumulation of microglia specifically near injured CNS axons, an event implicated in the maintenance and progression of a number of chronic neuroinflammatory and neurodegenerative diseases.

Referring to FIG. 1, a preferred fabrication process flow for design of a compartmentalized microfluidic system, according to one embodiment of the present invention, is shown. The microfluidic system, which can function as a co-culture platform, is preferably fabricated in poly(dimethylsiloxane) (PDMS), a biocompatible silicone rubber, following well established replica molding procedures. Preferably, the device mold is constructed using standard SU-8 photolithography, and preferably involves a two-layer microfabrication process. Initially, at step 110, the fabrication process begins with a bare silicon wafer (e.g., obtained from University Wafer, MA). As depicted at step 120, an initial thin-film photoresist layer 123 (e.g., SU-8 2002; height=2.5 microns) is spun, soft baked, and optically exposed. Subsequently, at step 130, the substrate is post-exposure baked and immersed in developer to define a circular array of microchannels 133. In other words, during these intermediate steps of the process, the silicon wafer is coated with the thin-film photoresist layer 123 (e.g., SU-8 2002, Microchem, MA), soft baked, exposed with a high resolution DPI transparency mask (Cad/Art, OR), and developed to define the circular array of microchannels 133. The microchannels preferably have the following dimensions: height from about 2 microns to about 4 microns; width from about 8 microns to about 10 microns; length of about 500 microns; and minimum spacing from about 15 microns to about 25 microns. Subsequently, referring to steps 140 and 150, a thick-film photoresist 143 (e.g., SU-8 3050; height=150 microns) is processed similarly to define larger fluidic access ports 148, which are shown relative to the array of microchannels 133. The thicker photoresist 143 (SU-8 3050; e.g., obtained from Microchem, MA) is thus used to define the multiple axonal (outer port; diameter of preferably about 4 mm) and somal (inner port; diameter of about 3 mm) compartments (each compartment having a height of preferably about 150 microns to about 200 microns). Exemplary details pertaining to photoresist (soft/hard/post exposure) bake, exposure, and development times can be found in manufacturer's technical sheets (e.g., available from. Microchem, MA; incorporated herein by reference).

Thereafter, reproducible replication of the compartmentalized microfluidic device was done by soft polymer casting using, e.g., Sylgard 184 PDMS (Dow Corning, MI), as depicted at step 160. After replication at step 160 using poly(dimethylsiloxane) (PDMS) (depicted by reference numeral 165), a biocompatible silicone rubber, in which replica molding procedures are employed, the finished compartmentalized microfluidic device is customized with an array of fluidic access ports 178, e.g., through the use of commercially available dermal biopsy punch tools, as depicted at step 170. In one example, after replication, access ports 178 are formed using commercially available punch tools (Huot Instruments, WI).

The finished compartmentalized microfluidic device can thereafter be used for various experiments or studies that involve, for example, cell loading, co-culture studies, etc. For instance, as described in Example 1 herein (in the Examples section of the detailed description herein), prior to cell loading, replicated devices can be sonicated (Branson Ultrasonics, CT) in 70% ethanol for 5 min and dried with compressed air to remove PDMS debris and other surface contaminants. Cleaned devices are then placed feature-side down onto 40 mm glass bottom petri dishes (Willco Wells, Netherlands) and sealed upon contact. If a tighter seal is required, both the device and glass substrate can be exposed to a low-power (e.g., 25 W) oxygen plasma treatment for about 1 min (Harrick Plasma, NY) prior to contact. Devices can also be sterilized with, e.g., 100% ethanol, washed with doubly deionized water (e.g., ddH20; Millipore, MA) to remove residual ethanol, and coated with poly-D-lysine hydrobromide (e.g., 100 micrograms per ml; Sigma, Mo.). The finished compartmentalized microfluidic devices can then be stored, e.g. at 37 degrees C., until needed for experimentation.

The microchannels 133 can be designed with any shape, dimensions (length, width, depth), and surface characteristics (rough, smooth, or of varying surface topography) as needed or desired. According to one exemplary embodiment, the width of each microchannel 133 is preferably from about 3 microns to about 0.10 microns, and more preferably each microchannel 133 is about 8 microns in width. The height of each microchannel 133 is preferably from about 1 micron to about 5 microns, and more preferably each microchannel is about 2.5 microns in height. Moreover, the length of each microchannel 133 is preferably from about 500 microns to about 5000 microns in length.

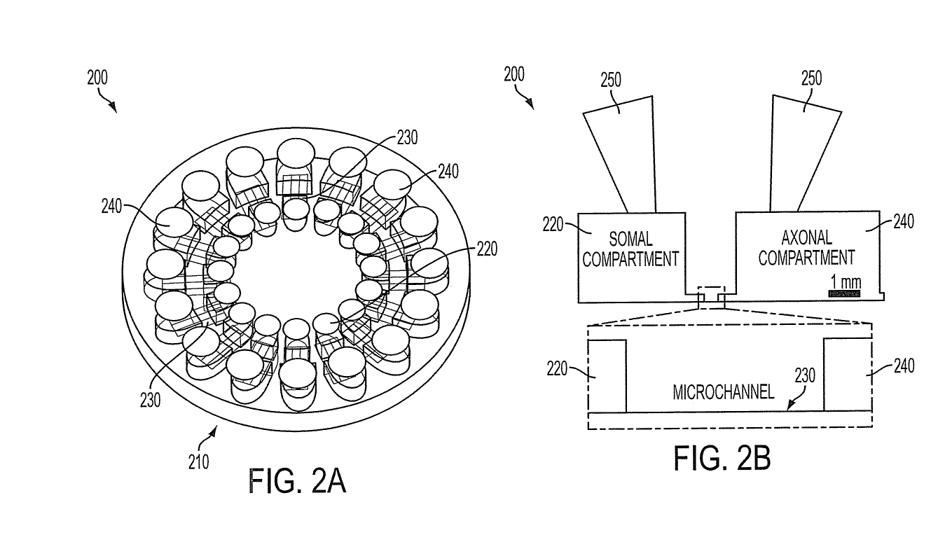

Referring to FIG. 2A, a schematic of a compartmentalized PDMS device 200 according to one embodiment of the present invention is shown to aide in visualization of the microchannels 230, somal compartments 220 and axonal compartments 240. Referring to FIG. 2B, a schematic diagram illustrates an expanded view of a microchannel 230, somal compartment 220 and axonal compartment 240. Each somal compartment 220 and axonal compartment 240 can be designed to any shape and size (length, width and depth) as needed or desired. In a preferred embodiment, each of the somal compartment 220 and axonal compartment 240 is preferably greater than about 3 mm in width. Larger size compartments can also be designed, for instance, to further enable pipetting close (e.g., less than 500 microns) to the microchannels 230 (pipets are shown schematically by reference numerals 250).

The compartmentalized PDMS device according to the present invention can be used, for example, for quantifying cell distribution on the somal side of the microchannels 230, e.g., after seeding the somal compartments 220 with neurons. Depending on the cross sectional area of the microchannels 230, cell migration through the microchannels 230 can be allowed or restricted under normal culture conditions.

The compartmentalized PDMS devices, for instance, as shown schematically in FIGS. 2A and 2B, can be spun, e.g., at 600 rpm (4.5 g) for 1.5 min to further enhance cell proximity to the microchannel-somal compartment interface. In one set of experiments, cell placement within the first 20 microns of the microchannel interface was increased 13-fold in spun devices as compared to control (n=10 replicates). In separate experiments, devices containing low (120 cells per mm), medium (300 cells per mm), and high (1500 cells per mm) density neurons were either left stationary or immediately spun to enhance cell position near the microchannel interface. After 7 days, neurite outgrowth was quantified for all conditions. Spinning resulted in up to 2-fold increases in axon throughput, or number of channels containing axon processes, in low (n=5) and medium density cultures (n=5). Under high seeding conditions (n=5), no enhancement could be observed, but nearly 100% of the channels contained neurites as shown by CalceinAM staining. Alignment was also achieved in the axonal compartment as shown by the utilization of a PDMS wedge near the microchannel inteface. Microglia were distributed along the axis of the wedge and the device was immediately spun to align the microglia along the edge of the wedge. After removal of the PDMS piece, a band of microglia cells remained.

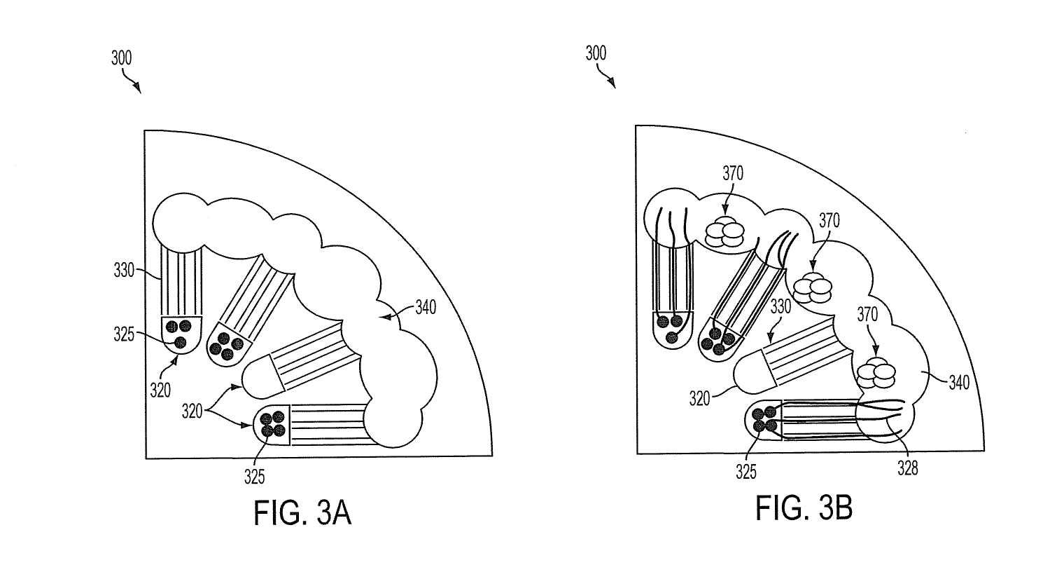

FIG. 3A depicts a portion of a compartmentalized device 300 configured so that it consists of four somal compartments 320 connected to a unified axonal compartment 340 via an array of microchannels 330. Neurons 325 were added to three of the four somal sectors (one intentionally left empty as a negative control.

The compartmentalized device 300 can be used for studying cell-cell interactions, for instance, microglial response to degenerating axons. Referring to FIG. 3B, the schematic diagram of the compartmentalized device 300 depicts primary microglial cells 370 selectively placed between bundles of axon outgrowth 328 in the unified axonal compartment 340, e.g., through the use of PDMS microstencils. Microchannels are depicted by reference numeral 330. In one set of experiments, neurons 325 were treated with control or peroxide containing media (2 mM) and volumes were adjusted to prevent diffusion into the axonal compartment. Chemotaxis and accumulation of microglia in response to signals from (i) no axons, (ii) healthy axons, or (iii) degenerating axons was quantified by measuring the total displacement (net upward or downward movement) of microglia along the microchannel interface in the axonal compartment 340 prior to, 48 h after, and 72 h after insult with respect to their initial seeding position. The compartmentalized system of the present invention preferably consists of independent units of radial microchannel arrays that fluidically isolate somal from axonal compartments. Fluidic access ports punched near the microchannels allow for direct pipetting of cells into the device. Adjacent somal or axonal compartments can be readily merged so that independent groups of neurons or axons can be maintained in either separate or uniform microenvironments.

According to a preferred embodiment, the configuration of the microfluidic device design is a circular platform consisting of a plurality of independent units. Each unit is defined by an axonal and somal compartment that are physically interconnected by an array of microchannels. These independent units are arranged radially in a closed circular pattern and are separated from each other by a minimum spacing of about 250 microns. Neurons are seeded into the somal compartment, and as axons extend they are passively guided through the microchannels and into the axonal compartment. Using this configuration, multiple unique experiments can be performed within the same device.

As proof of principle, the present inventors labeled consecutive axonal compartments with differing dyes, and demonstrated that retrograde labeling of populations of neurons is confined to each distinct somal compartment. Additionally, adjacent somal or axonal compartments can be readily merged, resulting in device configurations that differ in size and connectivity of the neuronal and axonal microenvironments. Exemplary configurations of the compartmentalized device include, for instance: (1) fully segmented somal and axonal compartments; (2) fully merged somal and axonal compartments; (3) fully merged somal and fully segmented axonal compartments; and (4) fully segmented somal and fully merged axonal compartments. Depending upon experimental needs, minor permutations to these different configurations can yield an even greater number of device possibilities by simply merging neighboring compartments with commercially available punch tools during the creation of fluidic access ports.

### Neuronal and Glial Cell Positioning

Cell positioning can be modulated by several distinct methods in our platform, allowing for versatility when developing novel axon-glia co-culture studies. While previous attempts have been made to utilize microchannels to guide neurites and establish co-culture conditions, no previous device has (a) integrated large access ports within close proximity to microchannel features to provide straightforward loading of cells near guidance features, (b) allowed incorporation of micropatterning techniques (e.g., microcontact printing, or microstencils) to manipulate co-culture conditions, and (c) optimized platform geometry so that centrifugal forces can be quickly applied to provide a robust mechanism to manipulate neuronal and glial cell distribution.

### Neuronal Cell Positioning

In the culture platform of the present invention, it is preferred that large fluidic ports are incorporated within close (e.g., <500 microns) proximity to the microchannels. This surprisingly enables the ability to pipette cells, analytes, and other chemical reagents directly, without requiring traditionally lengthy microfluidic input and output channels. When introduced into the device with a standard pipette, cells can consistently be placed within about 20 to 30 microns of the microchannel features. As a result, the numbers of neurons extending axons through the microchannel features is maximized, with a very high percentage (nearly 100%) of channels filled with numerous axons, e.g., after 7 days.

### Centrifugation

The circular geometry of the platform lends itself to centrifugation, or spinning, to enhance cell position with respect to physical features of the device. Although spinning has been previously shown to modulate cell placement, the circular device of the present invention enabled rapid centering and centrifugation of the device within 2 minutes of seeding cells. Thus, even highly adherent cells such as neurons can be effectively and uniformly centrifuged. Compared to non-spun devices, centrifuged devices in certain studies surprisingly showed a 13-fold enhancement in neuronal cell placement within about 20 microns of the microchannel interface with only 4.5 g of force applied, the lowest among reported devices. Furthermore, with the exception of high seeding densities, centrifugation resulted in up to a two-fold increase in the number of channels containing neurite processes.

Additionally, a novel approach using centrifugal forces can be exploited to align glial cells adjacent to physically imposed structures, such as PDMS wedges. The advantage of this approach is that glia can be compacted into a very thin (e.g., <350 microns) band near axonal processes and blocked from traversing into unwanted regions of the compartment. Cell viability, as assessed by calcein AM uptake and by trypan blue staining, was not adversely affected by centrifugation over the range tested (0-1200 rpm), as compared to non-spun cells (data not shown), and cells continued to remain healthy in the device for over 3 weeks under standard media conditions.

### Co-Culture

The microfluidic platform of the present invention also allows directed coculture of glial cells within microdomains of the axonal compartment. Since the device employs large access ports, PDMS microstencils can be used to directly place glial cells within a defined microenvironment in the multi-compartment platform. As a result, populations of glial cells can be placed in defined regions either adjacent to or in between areas of axons.

If intimate axon-glia co-culture is required, glial cells can be reproducibly pipetted along the axonal-microchannel interface, in large part due to directed flow and capillary action. This results in a large percentage of microglia in intimate contact with axons, e.g., where over 93% of microglia are in intimate contact with axonal processes. The co-culturing procedure does not appear to adversely affect axonal viability, as no observable markers of axonal degeneration (e.g. increase in number of blebs or changes in axon morphology) were seen in experiments performed. Additionally, the cellular distribution of microglia in the axonal compartment was unaffected by the presence of axons, as the microglia distribution was similar in the absence of axons (data not shown).

### Microglial Response to Axonal Degeneration

The multicompartment platform of the present invention was utilized to determine whether microglia migrate specifically toward injured CNS axons. Accumulating evidence suggests that recruitment of microglia during CNS inflammatory disease can contribute to neurodegeneration, although it is unknown whether such recruitment occurs via direct axon-microglial interactions. To answer this question, the device was configured such that four consecutive axonal compartments were merged to allow sites for microglial stencil patterning near axons. Neurons were plated in the somal compartments, and centrifuged (v=600 rpm, t=90 s) to enhance axonal throughput. After dense bundles of axons arising from distinct somal compartments had extended into the unified axonal compartment, primary rat microglia were directly placed in between areas of axonal outgrowth. By placing microglia in defined regions within the device, a common starting point for cellular migration could be assessed. To induce oxidative stress, which has been implicated in the pathogenesis of many neuroinflammatory diseases including multiple sclerosis and human immunodeficiency virus (HIV) dementia, we added H₂O₂ selectively to the somal compartment of only one group of neuronal cell bodies. This resulted in induction of neuronal cell death and accompanying axonal degeneration over the course of 24-72 h. Quantification of microglial chemotaxis and accumulation demonstrated that, over the course of 72 h, microglia migrated an average of 70±21 microns (mean±standard error; n=4) when placed in areas devoid of axons, reflecting basal rates of migration. Migration distance was increased when microglia were placed adjacent to healthy axons, demonstrating endogenous levels of migration under standard cell culture conditions. Strikingly, however, microglia migration toward degenerating axons was further enhanced two-fold on average as compared to healthy axons, demonstrating preferential accumulation near degenerating, as compared to healthy, axons.

The precise positioning of cells between independent groups of CNS axons in the multi-compartment platform surprisingly allows one to investigate microglial responses to degenerating axons. While standard culture platforms and commercially available kits can quantify cellular chemotaxis, they cannot address whether microglial accumulation occurs specifically in response to CNS axonal injury. With the present invention, it was found that microglia preferentially accumulate near degenerating, as compared to healthy, axons. Importantly, the absence of neuronal cell bodies or other glial cells in the fluidically isolated axonal compartment implies that microglia respond specifically to axon-derived signals. Such signals may either be soluble or membrane-bound, and the identities of these molecules as well as the microglial receptors that recognize these ligands remain to be determined. Further investigations with the microfluidic platform of the present invention will allow for the dissection of the molecular mechanisms of microglial chemotaxis and accumulation in response to degenerating axons.

The device of the present invention can be easily configured to investigate other aspects of axon-glia interactions. For example, one device configuration, in which neurons residing within a uniform microenvironment give rise to groups of axons that can be independently manipulated, would be particularly suited to study axon-specific responses to a variety of chemicals, toxins, and glia co-culture interactions. Alternatively, the device can be configured to yield a single large somal and axonal compartment which can take full advantage of the large array of microchannels present in the device to collect extensive data on a specific axon-glia interaction.

Moreover, in accordance with the present invention, co-culturing conditions can be readily modulated depending upon the biological question. The present invention has demonstrated the utility of loading small numbers of glial cells in spatially defined areas within the device. However, if high co-culturing efficiencies are needed, for example, glia can be directly loaded along the axonal compartment-microchannel interface. The close contact of such a high percentage of co-cultured cells with axons in the device lends itself to a variety of other applications including, for instance, the study of gene expression changes in glial cells interacting with axons, as well as the development of robust in vitro CNS myelination systems.

The multi-compartment co-culture platform, in accordance with the present invention, represents an innovative approach to studying axon-glia interactions. By virtue of the circular configuration of the platform, large radial array of microchannels, compartment configurability, and ports within close proximity to microchannel features, many surprisingly advantageous device characteristics were realized including, but not limited to, (a) the use of centrifugal forces to modulate placement of both neurons and glial cells, (b) fluidic isolation of large numbers of axonal processes, (c) glial micropatterning, and (d) intimate axon-glia co-culturing. Additionally, the circular geometry requires minimal external forces to enhance cell placement within the device, thereby maximizing cell viability and providing new techniques to co-culture glia with axons. The microfluidic platform of the present invention was utilized to demonstrate, inter alia, preferential accumulation of microglia specifically to injured as compared to healthy axons, serving as a foundation to elucidate mechanisms of axon-glia interactions in neurological disease maintenance and progression. The multi-compartment co-culture platform also surprisingly enables distinct modes of axon-glia co-culture and provides experimental versatility to investigate axon-specific and axon-glia-specific cellular and molecular events implicated in neurobiological disease.

### EXAMPLES

Representative embodiments of the present invention are further illustrated in the following examples. These are provided by way of illustration and are not intended in any way to limit the scope of the invention.

### Example 1

### Fabrication of Co-Culture Platform

As described herein, with reference to FIG. 1, a co-culture platform was fabricated in poly(dimethylsiloxane) (PDMS), a biocompatible silicone rubber, following well established replica molding procedures. Briefly, a master template was created using a two-layer microfabrication process. Silicon wafers (University Wafer, MA) were coated with a thin photoresist layer (SU-8 2002, Microchem, MA), soft baked, exposed with a high resolution DPI transparency mask (Cad/Art, OR), and developed to define a circular array of 1500 microchannels (dimensions: height=2-4 microns, width=8-10 microns, length=500 microns, and minimum spacing=15-25 microns). The process was immediately repeated with a thicker photoresist (SU-8 3050, Microchem, MA) to define multiple axonal (outer port; diameter=4 mm) and somal (inner port; diameter=3 mm) compartments (height=150-200 microns). Photoresist (soft/hard/post exposure) bake, exposure, and development times were followed according to standard manufacturer's technical procedures (Microchem, MA). Reproducible replication of the device was done by soft polymer casting using Sylgard 184 PDMS (Dow Corning, MI). Once replicated, access ports were formed using commercially available punch tools (Huot Instruments, WI). Prior to cell loading, replicated devices were sonicated (Branson Ultrasonics, CT) in 70% ethanol for 5 min and dried with compressed air to remove PDMS debris and other surface contaminants. Cleaned devices were placed feature-side down onto 40 mm glass bottom petri dishes (Willco Wells, Netherlands) and sealed upon contact. If a tighter seal was required, both the device and glass substrate were exposed to a low-power (25 W) oxygen plasma treatment for 1 min (Harrick Plasma, NY) prior to contact. Devices were sterilized with 100% ethanol, washed three times with doubly deionized water (ddH20; Millipore, MA) to remove residual ethanol, and coated with poly-D-lysine hydrobromide (Sigma, Mo.). Devices were stored at 37 degrees C. until needed for experimentation.

### Cell Preparation

Primary hippocampal neurons were derived from embryonic day 17 (E17) pups and resuspended. Primary microglial cultures were derived from postnatal rat pups. Briefly, dissociated hippocampal cells were plated in DMEM/F12/10% fetal bovine serum (FBS), and media was changed every 3 days. Between days 10 and 14, cells were shaken at 225 rpm for 90 min, and non-adherent cells were collected and plated. After 30 min, the microglia cells had attached to the plate, and non-adherent contaminating cells were removed. This resulted in a population of microglia of >95% purity. The BV2 microglial cell line, used in several co-culturing experiments, was maintained in DMEM/10% FBS. Devices were washed 3× with ddH₂0, filled with serum-containing media, and placed in a standard humidified cell culture incubator set to 37 degrees C. and 5% CO₂ (Thermo Scientific, MA) for 15-30 min. Primary neurons were loaded into the somal compartment of the device in increments of 5 microliters. If spinning was required, devices were moved to a spinner (Laurell Technologies, MA) immediately after cell loading, centered, and spun for 1-2 min at 400-1200 rpm. For experiments in which cells were labeled with a fluorescent protein, dissociated neurons were nucleofected (Amaxa, MD) with a plasmid encoding the TdTomato gene as per the manufacturer's instructions. Efficiency of labeling was greater than 70%.

### PDMS Stencil Patterning

PDMS stencils were prepared from unpatterned 3 mm PDMS slabs that were cut to yield thin 500-1000 micron membranes. Circular feature sizes (diameters) ranging from 500 microns to 1000 microns were then created by piercing the membrane with surgical needles (Fisher Scientific, PA). The stencils were sonicated with ethanol, dried, and placed within the device prior to cell seeding. During cell placement, glial cells were first concentrated and then slowly pipetted into the cavity of the PDMS stencil. After 1 hour, the stencil was carefully removed and media was filled into the compartment without noticeable cell dispersion.

### Restriction of Diffusing Analytes

To delineate the effects of inflammatory mediator treatment on either the cell body or axon, fluidic isolation was established to prevent the diffusion of analytes from one compartmentalized region of the neuron to the other. Fluid volumes were modified such that the somal compartment was of lower fluidic height than the axonal compartment. By doing so, a hydrostatic pressure was established between these microchannel-connected compartments, thereby creating a small flow to counteract diffusive forces. A 1 microliter bolus of fluorescein isothiocyanate (FITC; Sigma, Mo.) dye was introduced to the somal compartment and was imaged over the course of 24 h.

Empirically, a fluid height difference greater than or equal to 2 mm was sufficient to prevent the diffusion of a low-molecular weight (MW 700 Da) analyte to the axonal compartment for at least 24 h. In principle, the ideal pressure required to maintain fluidic isolation could be determined analytically, however, empirical testing provides proof of principle that fluid isolation could be achievable for the desired pressures, times and proposed analytes. Continued maintenance of the height differential was achieved by adding 5 microliters of media to the higher volume compartment daily. This allowed for restriction of small analytes (e.g. nitric oxide, MW 30 Da) over several days as verified by commercially available kits (Griess nitric oxide detection kit; Promega, Madison, Wis.).

### Imaging and Analysis

Images were obtained in a live-cell Zeiss inverted microscope (Axio Observer) using Zeiss Axiovision software. After data collection, images were exported to either Metamorph (MDS Analytical Technologies, Toronto, Calif.) or NIH ImageJ (Bethesda, Md.) for further analysis. Statistical analysis was then performed.

### Surface Density

The number of cells within a given surface area was measured under 10× magnification utilizing the grid function of Metamorph which overlays a two-dimensional mesh of user-defined size on top of the image. Bins of defined widths (10 or 50 microns) and varying lengths were used to calculate cell distribution and density.

### Microglia Chemotaxis and Accumulation.

Microglia were patterned within a circular microstencil (diameter=500 microns) in an area between arrays of microchannels. After microglia attached (approximately 1 hour later), the microstencil was carefully removed to minimize cell dispersion. Microglial movement was quantified parallel to the microchannel interface (arbitrarily defined as the y-axis) and information pertaining to cellular position in the orthogonal direction (x-axis) was not recorded. The furthest microglia (n=6) along the y-axis but within 500 microns of the x-axis were quantified in terms of their average y-displacement from the start of the microchannels (y=0). For each time point and experimental condition, the same procedure was repeated to determine the average position of the furthest group of microglia under those circumstances. Microglia accumulation was defined as the net displacement along the y-axis between 0 and 72 h of the same experimental condition (no axons, healthy axons, or degenerating axons; n=4 independent experiments for each situation).

### Results

### Microfluidic Device Design

In this experimental study, the default configuration of the circular platform consists of sixteen independent units. Each unit is defined by an axonal (outer) and somal (inner) compartment that are physically interconnected by an array of 90 microchannels. These independent units are arranged radially in a closed circular pattern and are separated from each other by a minimum spacing of 250 microns. Neurons are seeded into the somal compartment, and as axons extend they are passively guided through the microchannels and into the axonal compartment. Using this default configuration, multiple unique experiments can be performed within the same device. As proof of principle, we labeled consecutive axonal compartments with differing dyes, and demonstrate that retrograde labeling of populations of neurons is confined to each distinct somal compartment. Additionally, adjacent somal or axonal compartments can be readily merged, resulting in four major device configurations that differ in size and connectivity of the neuronal and axonal microenvironments. These configurations consist of: (1) fully segmented somal and axonal compartments; (2) fully merged somal and axonal compartments; (3) fully merged somal and fully segmented axonal compartments; and (4) fully segmented somal and fully merged axonal compartments. Depending upon experimental needs, minor permutations to these four major configurations can yield up to 256 (16×16) device possibilities by simply merging neighboring compartments with commercially available punch tools during the creation of fluidic access ports.

### Neuronal and Glial Cell Positioning

Cell positioning can be modulated by several distinct methods in our platform, allowing for versatility when developing novel axon-glia co-culture studies. While previous attempts have been made to utilize microchannels to guide neurites and establish co-culture conditions, no previous device has (a) integrated large access ports within close proximity to microchannel features to provide straightforward loading of cells near guidance features, (b) allowed incorporation of micropatterning techniques (e.g., microcontact printing, or microstencils) to manipulate co-culture conditions, and (c) optimized platform geometry so that centrifugal forces can be quickly applied to provide a robust mechanism to manipulate neuronal and glial cell distribution.

### Neuronal Cell Positioning

In the culture platform of the present invention, it is preferred that large fluidic ports are incorporated within close (e.g., <500 microns) proximity to the microchannels. This surprisingly enables the ability to pipette cells, analytes, and other chemical reagents directly, without requiring traditionally lengthy microfluidic input and output channels. When introduced into the device with a standard pipette, cells can consistently be placed within about 20 to 30 microns of the microchannel features. As a result, the numbers of neurons extending axons through the microchannel features is maximized, with a very high percentage (nearly 100%) of channels filled with numerous axons, e.g., after 7 days.

### Centrifugation

The circular geometry of the platform lends itself to centrifugation, or spinning, to enhance cell position with respect to physical features of the device. Although spinning has been previously shown to modulate cell placement, the circular device of the present invention enabled rapid centering and centrifugation of the device within 2 minutes of seeding cells. Thus, even highly adherent cells such as neurons can be effectively and uniformly centrifuged. Compared to non-spun devices, centrifuged devices in certain studies surprisingly showed a 13-fold enhancement in neuronal cell placement within about 20 microns of the microchannel interface with only 4.5 g of force applied, the lowest among reported devices. Furthermore, with the exception of high seeding densities, centrifugation resulted in up to a two-fold increase in the number of channels containing neurite processes.

Additionally, a novel approach using centrifugal forces can be exploited to align glial cells adjacent to physically imposed structures, such as PDMS wedges. The advantage of this approach is that glia can be compacted into a very thin (e.g., <350 microns) band near axonal processes and blocked from traversing into unwanted regions of the compartment. Cell viability, as assessed by calcein AM uptake and by trypan blue staining, was not adversely affected by centrifugation over the range tested (0-1200 rpm), as compared to non-spun cells (data not shown), and cells continued to remain healthy in the device for over 3 weeks under standard media conditions.

### Co-Culture

The microfluidic platform of the present invention also allows directed coculture of glial cells within microdomains of the axonal compartment. Since the device employs large access ports, PDMS microstencils can be used to directly place glial cells within a defined microenvironment in the multi-compartment platform. As a result, populations of glial cells can be placed in defined regions either adjacent to or in between areas of axons.

If intimate axon-glia co-culture is required, glial cells can be reproducibly pipetted along the axonal-microchannel interface, in large part due to directed flow and capillary action. This results in a large percentage of microglia in intimate contact with axons, e.g., where over 93% of microglia are in intimate contact with axonal processes. The co-culturing procedure does not appear to adversely affect axonal viability, as no observable markers of axonal degeneration (e.g. increase in number of blebs or changes in axon morphology) were seen in experiments performed. Additionally, the cellular distribution of microglia in the axonal compartment was unaffected by the presence of axons, as the microglia distribution was similar in the absence of axons (data not shown).

### Microglial Response to Axonal Degeneration

The multicompartment platform of the present invention was utilized to determine whether microglia migrate specifically toward injured CNS axons. Accumulating evidence suggests that recruitment of microglia during CNS inflammatory disease can contribute to neurodegeneration, although it is unknown whether such recruitment occurs via direct axon-microglial interactions. To answer this question, the device was configured such that four consecutive axonal compartments were merged to allow sites for microglial stencil patterning near axons. Neurons were plated in the somal compartments, and centrifuged (v=600 rpm, t=90 s) to enhance axonal throughput. After dense bundles of axons arising from distinct somal compartments had extended into the unified axonal compartment, primary rat microglia were directly placed in between areas of axonal outgrowth. By placing microglia in defined regions within the device, a common starting point for cellular migration could be assessed. To induce oxidative stress, which has been implicated in the pathogenesis of many neuroinflammatory diseases including multiple sclerosis and human immunodeficiency virus (HIV) dementia, we added H₂0₂ selectively to the somal compartment of only one group of neuronal cell bodies. This resulted in induction of neuronal cell death and accompanying axonal degeneration over the course of 24-72 h. Quantification of microglial chemotaxis and accumulation demonstrated that, over the course of 72 h, microglia migrated an average of 70±21 microns (mean±standard error; n=4) when placed in areas devoid of axons, reflecting basal rates of migration. Migration distance was increased when microglia were placed adjacent to healthy axons, demonstrating endogenous levels of migration under standard cell culture conditions. Strikingly, however, microglia migration toward degenerating axons was further enhanced two-fold on average as compared to healthy axons, demonstrating preferential accumulation near degenerating, as compared to healthy, axons.

### Example 2

### Compartmentalized Microfluidic Culture Platform to Study Mechanism of Paclitaxel-Induced Axonal Degeneration

Chemotherapy induced peripheral neuropathy is a common and dose-limiting side effect of anticancer drugs. Conventional studies aimed at understanding the underlying mechanism of neurotoxicity of chemotherapeutic drugs have been hampered by lack of suitable culture systems that can differentiate between neuronal cell body, axon or associated glial cells. An in vitro compartmentalized microfluidic culture system was developed to examine the site of toxicity of chemotherapeutic drugs. To test the culture platform, paclitaxel was used, since this drug is a widely used anticancer drug for breast cancer, and because it causes sensory polyneuropathy in a large proportion of patients and there is no effective treatment. Paclitaxel, a diterpene alkaloid drug, is a commonly used chemotherapeutic agent against breast, lung and ovarian cancer. One of the major dose-limiting side effects is distal axonal, mainly sensory, polyneuropathy. In previous conventional in vitro studies, paclitaxel induced distal axonal degeneration but it was unclear if this was due to direct toxicity on the axon or a consequence of toxicity on the neuronal cell body.

In the study described herein, the underlying mechanism of paclitaxel-induced axonal degeneration was investigated through the use of the microfluidic platform of the present invention that allowed for physically and fluidically isolating cellular compartments, as well as to gauge the protective role of recombinant human erythropoietin. Since most polyneuropathies are “dyingback” neuropathies, this study sought to determine if paclitaxel caused the most degeneration when applied to the distal axon as compared to the cell body.

### Materials and Methods

### Microfabricated Chamber Preparation

A two-step photolithographic process was utilized to create the master mold. Silicon wafers (University Wafer, MA) were coated with SU-8 2002 (Microchem; MA), spun, and soft baked using parameters specified by the manufacturer to yield a resist thickness of 2.5 microns. An array of microchannels, each with dimensions: width=10 microns, length=500 microns, were defined by UV light exposure through a high resolution DPI transparency (Cad/Art, OR). The exposed substrate was once again baked, to enhance polymer cross-linking post exposure, and developed as stated in the resist technical sheet to fully define the microchannels. The process was immediately repeated with SU-8 3050 (Microchem; MA) to define the fluidic reservoirs with dimensions: width=3 mm, length=13 mm. The master mold was then treated with trichlorosilane (United Chemical Technologies; PA) for 30 min to create a nonstick surface for subsequent processing. Standard soft lithography was performed using Sylgard 184 polydimethylsiloxane (PDMS) (Dow Corning, MI). After curing, the PDMS was carefully removed from the master and access ports were created using a suite of dermal biopsy punch tools (3-6 mm) (Huot Instruments, WI).

### Cell Preparation

All experiments involving animals were conducted according to protocols approved by the Animal Care and Use Committee of the Johns Hopkins University School of Medicine. Unless otherwise noted tissue culture supplies were obtained from Invitrogen (Carlsbad, Calif.). Dorsal root ganglia (DRG) neuronal cultures were prepared. Briefly, DRGs were dissected from decapitated embryonic age day 15 rats. Once obtained, cells were enzymatically dissociated with 0.25% Trypsin in L15 medium and then suspended in media. The DRGs were maintained in Neurobasal medium containing 10% fetal bovine serum, 20% glucose, 1% penicillin/streptomycin, B-27 supplement, 2 M L-glutamine, and 10 ng/ml glial derived nerve growth factor (GDNF). Two days after seeding cells, neurobasal media containing 10 μM of Cytosine arabinoside was added to the cultures in order to decrease the amount of glial cells. Paclitaxel (Sigma-Aldrich) was dissolved in cremophor EL/ethanol (50/50 v/v) for a stock concentration of 5.0 mg/ml and stored at −20° C. Recombinant human erythropoietin (EPO) was obtained from R & D Systems (Minneapolis, Minn.) and dissolved in phosphate buffered saline and stored at −20° C.

DRG neurons were loaded into the soma side of devices and grown for 5-7 days to allow axons sufficient time to grow through channels and into the axonal side at a sufficient length. Paclitaxel was diluted in neurobasal media to achieve a concentration of 25 ng/mL and applied to either the neuronal cell body or axonal side. EPO was diluted in culture medium and applied to neuronal cell body or axonal side. Cells were subsequently stained with calcein AM (green) at a concentration of 2.5 μM for 1 h and imaged using a fluorescent microscope.

### Results

In order to identify the susceptibility of axons and cell bodies to paclitaxel, we added 25 ng/mL of paclitaxel to either axon or cell body chambers and continued to culture the DRGs for another 24 hours. Once images of the fluorescently labeled cells and axons were captured, we used ImageJ to calculate axon lengths and calculated percent change in axon length compared to 24 hours before taxol exposure. It was observed that paclitaxel caused axonal degeneration when applied to the axonal compartment, but not when applied only to the cell body compartment.

Studies on the neuroprotective effect of EPO were performed with concurrent administration of paclitaxel, which was applied to either the neuronal cell body or axonal side. In order to study the effect of EPO on different cellular compartments, we also applied the hormone to either the neuronal cell body or axonal side of the chambers. It was observed that there is not a large difference in axon morphology or length, demonstrating the neuroprotective effect of EPO as it seems to prevent axon degeneration. This effect was also seen when EPO was applied to the neuronal cell body compartment when paclitaxel was applied to the axonal compartment. Axon degeneration was defined as the change in axon length over the total axon length expressed as a percentage. When only paclitaxel was applied to the axonal side, we saw a 28.6+11.2% decrease in the length of axons, compared to a control in which no paclitaxel was added that showed a 0.42+0.25% increase in axon length. Paclitaxel applied in combination with EPO on the axon side showed a 1.02+0.42% decrease in axon length, which was more comparable to the control than to the paclitaxel-induced degeneration condition. When paclitaxel was applied on the axon side while EPO was applied to the soma side, we saw a 1.34+0.59% decrease in axon length, again demonstrating a neuroprotective effect.

### Discussion

Paclitaxel-induced sensory neuropathy is a frequent and disabling side effect, and can potentially lead to the discontinuation of chemotherapy. The microfluidic platform used in this study allowed for better clarifying the mechanism for paclitaxel-induced degeneration. The device used in this study does not allow mixture of culture fluids between chambers and provide glass substrate for better optical microscopy compared to Campenot chambers and its derivatives. Furthermore, the microfluidic platform is based on the PDMS, which has excellent gas exchange properties. The microfluidic platforms used in this study are simple to load with sufficiently high-density cells and corresponding higher amounts of axons inside microchannels. There was ample segregation of neuronal cell bodies and axons.

This study clearly shows that paclitaxel causes axon degeneration through local mechanisms, but that this local toxicity can be controlled by intracellular events induced at the cell body as shown by the EPO data. Paclitaxel induces an increase in detyrosinated tubulin, thereby leading to cold-stable microtubule assembly within the axons. How this leads to axonal degeneration is still unknown, although prevailing hypothesis is that it interferes with axonal transport depriving distal axons of their vital nutrients and cellular substrates. Rapid degeneration seen in previous studies and in our culture system suggests perhaps a different mechanism. An observation in the axonal chambers in this study was the presence of axonal blebbing even in the most proximal segments of axons in paclitaxel-treated axonal chambers. Axonal blebbing is often regarded as a prelude to axonal degeneration but it can be a reversible process. Local axonal activation of protein degradation pathways, such as caspases or calpains could lead to axonal blebbing and eventual degeneration.

The microfluidic chamber platform, in accordance with the present invention, was used to examine the site of axon protective action of EPO, expecting that local axonal application of EPO would prevent axonal degeneration induced by paclitaxel. We, however, found that EPO was able to prevent paclitaxel-axonal degeneration even when it was applied to the cell body, away from the axon and paclitaxel. Although the mechanism of this axon protection is unknown, it is possible that EPO-induced changes in intracellular signaling events are transported down the axon using fast axonal transport and block the toxicity of paclitaxel. We do not know if this type of potential mechanism of neuroprotection may apply to other axon protective therapies, but if it does, then the implications for drug development for peripheral neuropathies are immense. One of the limitations of developing therapies for nervous system indications is that axons and neurons are behind a blood-brain/nerve-barrier. This requires that the drugs be able to cross the blood-brain-barrier. However, there are exceptions to this rule and the blood-brain-barrier within the dorsal root ganglia is very leaky. If axon protection can be achieved by action of a drug on the neuronal cell body, even for toxins that cause local axonal degeneration, there would be a less stringent requirement for the drug to cross the blood-brain-barrier. Future studies will help us define if this is a general principle.

This study demonstrates that the microfabricated platform, composed of a microfluidic culture system, is robust, easy to manufacture and reliable. It allows separation of axons from neuronal cell bodies and independent manipulation of each compartment. It can be used to study mechanisms of axonal degeneration, protection against axonal degeneration and developmental events such as myelination. Furthermore, the manufacturing process is scalable to generate templates with more than hundred chambers that can be independently manipulated, thus allowing high-content studies including drug screening. Through the use of this device, it was demonstrated that paclitaxel causes degeneration of axons through local mechanisms. It was also shown that this effect can be counteracted through the administration of EPO both at the cell body and at the axon, indicating implications for drug development for polyneuropathies. This compartmentalized microfluidic culture system can be used for studies aimed at understanding axon degeneration, neuroprotection and development of the nervous system.

While the above description of the present invention contains many examples, these should not be construed as limitations on the scope of the invention, but rather should be viewed as exemplifications of preferred embodiments thereof. Many other variations are possible. Accordingly, the scope of the invention should be determined not by the embodiments illustrated, but by the appended claims.

What is claimed is:

1. A compartmentalized microfluidic system, comprising:
a base substrate;
a plurality of microchannels;
a plurality of somal compartments; and
a plurality of axonal compartments,
wherein the microchannels are formed within the base substrate, and further wherein the microchannels are connected with the somal compartments and the axonal compartments.
